(19) United States
(12) Patent Application Publication
(10) Pub. No.: US 2013/0280770 A1
(43) Pub. Date: Oct. 24, 2013

(54) Transgenic Algae with Enhanced Oil Expression

(71) Applicants: Holly Loucas, Poris (CA); Tzann-wei Wang, Waterloo (CA); John Thompson, Waterloo (CA)

(72) Inventors: Holly Loucas, Poris (CA); Tzann-wei Wang, Waterloo (CA); John Thompson, Waterloo (CA)

(73) Assignee: Senesco Technologies, Inc., Bridgewater, NJ (US)

(21) Appl. No.: 13/642,768

(22) PCT Filed: Apr. 22, 2011
(86) PCT No.: PCT/US11/33585

§ 371 Date: Jul. 8, 2013

Related U.S. Application Data
(60) Provisional application No. 61/326,979, filed on Apr. 22, 2010.

(51) Int. Cl. C12P 7/64; C12N 15/82

(52) U.S. Cl. CPC C12P 7/649; C12N 15/8247
USPC 435/134; 435/257.2; 435/471

(57) ABSTRACT

The present invention provides transgenic algal cells that produce an increased amount of oil, methods of making transgenic algal cells, and methods of obtaining biofuel from the transgenic algal cells.

### BACKGROUND

The sustainable production of renewable energy is becoming an important goal of government and industry. First generation biofuels, produced mainly from food crops, are limited in their ability to achieve targets for biofuel production, climate change mitigation and economic growth (Mata (2010) Renewable and Sustainable Energy Reviews 14: 217-232). Thus, interest in second generation biofuels, produced from non-feedstocks including algae, has increased. The most common biofuels are biodiesel and bio-ethanol, which can replace diesel and gasoline, respectively, in today's cars with little or no modification to vehicle engines. They can also be produced using existing technologies and be distributed through the available distribution system. Algae has the advantage of not only oil production but also much higher energy yields per hectare, does not require agricultural land, and can be combined with pollution control, in particular with biological sequestration of CO₂ emissions and other greenhouse gases, or wastewater treatment (Mata (2010) Renewable and Sustainable Energy Reviews 14: 217-232). The main constraint of using algae for biofuel production is the cost. Large-scale cultivation of algae must have carefully controlled conditions and optimum nurturing environments in order to produce maximum growth resulting in maximum oil harvest. Setting up a system to incorporate pollution control such as sequestering CO₂ from flue gas emissions or waste water remediation processes and/or extraction of high value compounds for application in other process industries increases the economic potential.

In plants and animals, eukaryotic translation initiation factor 5A (eIF-5A), deoxyhypusine synthase (DHS) and deoxyhypusine hydroxylase (DHH) play a key role in cell growth and cell death. In plants, altered expression of either eIF-5A or DHS results in plants that grow faster producing larger overall plants and increased seed production with no change in oil composition (Wang (2005) Physiologia Plantarum 124: 493-503). Another positive effect of altered eIF-5A or DHS expression in plants is their ability to tolerate or recover from a wide range of stresses (Wang (2001) J. Biol. Chem. 276: 17541-17549, (2003) Plant Mol. Biol. 52: 1223-1235, (2005) Physiologia Plantarum 124: 493-503). Algae is an ideal organism to produce oil for biodiesel and if altered expression of either or both of these genes results in an increase in cell number it would also result in increased oil production while maintaining oil composition. One of the critical factors in using algae for biofuel production is the use of large-scale bioreactors, which require careful monitoring of growth conditions to maintain maximum algal growth. Any alteration in these conditions would result in a ‘stress’ environment and thus, would have a negative impact on algal growth rate. Having an alga that can tolerate stress or can recover faster after a stress has been imposed would increase the yield potential and thus, decrease oil production costs to more marketable levels.

### SUMMARY OF THE INVENTION

The present invention provides a transgenic algal cell that produces an increased amount of oil as compared to the amount of oil produced by a corresponding naturally occurring algal cell. The transgenic algal cell overexpresses a protein that contains hypusine. The transgenic algal cell may overexpress eukaryotic translation initiation factor 5A (eIF-5A), deoxyhypusine synthase (DHS), deoxyhypusine hydroxylase (DHH), or a combination thereof.

The eIF-5A protein may be obtained from any source. The eIF-5A protein may comprise an amino acid sequence having at least 85% sequence identity with SEQ ID NO: 4. The eIF-5A protein may be a poplar eIF-5A protein or any other plant eIF-5A protein. The eIF-5A protein may comprise an amino acid sequence as set forth in SEQ ID NO: 4.

The DHS protein may be obtained from any source. The DHS comprises an amino acid sequence having at least 85% sequence identity with SEQ ID NO: 6. The DHS protein may be a tomato DHS protein or any other plant DHS protein. The DHS protein may comprise an amino acid sequence as set forth in SEQ ID NO: 6.

The DHH comprises an amino acid sequence having at least 85% sequence identity with SEQ ID NO: 8. The DHH protein may comprise an amino acid sequence having SEQ ID NO: 8. In some embodiments, the DHH is encoded by a nucleotide sequence comprising SEQ ID NO: 7.

The present invention provides a method of producing transgenic algal cells that produce an increased amount of oil as compared to corresponding naturally occurring algal cells. The method comprises obtaining one or more constructs that encode one or more proteins that contain hypusine or that are involved in the expression or synthesis of a protein containing hypusine, transforming algal cells with the one or more constructs to obtain transgenic algal cells, cultivating the transgenic algal cells in a bioreactor under conditions and for a sufficient time to produce oil, and harvesting oil from the transgenic algal cells.

The algal cells may be transformed with two or more constructs, and each of the constructs may comprise the nucleic acid encoding eIF-5A, DHS, or DHH. The algal cells may be transformed with a construct comprising the nucleic acid encoding eIF-5A and a construct comprising the nucleic acid encoding DHS. Accordingly, the transgenic algal cells may contain the constructs encoding eIF-5A and DHS and overexpress eIF-5A and DHS.

The present invention provides constructs for expressing eIF-5A DHS, DHH, or a combination thereof. The construct may comprise a combination of two or more nucleic acids selected from the group consisting of nucleic acid encoding eIF-5A, nucleic acid encoding DHS, and nucleic acid encoding DHH.

The construct may comprise a nucleic acid encoding eIF-5A, DHS, or DHH operably linked to a promoter. The promoter may be the Saccharomyces cerevisiae glycolysis enzyme promoter. The construct may comprise the nucleic acid having a sequence as set forth in SEQ ID NO: 1, SEQ ID NO: 2.

The present invention provides a method of producing biodiesel fuel comprising growing transgenic algal cells that overproduce a protein that contains hypusine in a bioreactor under conditions and for a sufficient time to produce oil, harvesting oil from the transgenic algae cell, and processing the harvested oil into biodiesel fuel.

### BRIEF DESCRIPTION OF THE DRAWINGS

FIGS. 1A & B show TO line screen data at 4 days after initiation, 75% N-P-K nutrient, 3 reps/line, shaker with 30% shade, 120 rpm, % increase in growth rate of control at 75% BBM. (A) Upper: pPGK:PdF5A3cDNA-tNos construct (PF) (SEQ ID NO: 1). (B) Lower: pPGK:PdF5A3cDNA-tNos+pPGK:TDHS-tTEF1 double construct (FD) (SEQ ID NO: 2).

FIG. 2 shows CO₂ saturation and air recovery. Bubbling with CO₂ for 24 hours followed by bubbling with air for 24 hours, 100 μMol light, 3 reps/line, and 100% BBM. The constructs are: PF (PGK:PdF5A) and FD (PGK:PdF5A+PGK:TDHS).

FIG. 3 shows line screening data using a bioreactor and formula of media: 4× macro, 2×N, 2× micro, 24 hours growth, plus 60% CO₂, and 130 μMol light (3 reps/exp).

FIG. 4 shows oil production of algae in 4× macro, 2×N, and 2× micro, plus 60% CO₂, 130 μMol light after 24 hours growth in bioreactors (3 reps/exp).

FIG. 5 shows oil production of algae in 10× macro, 2×N, and 2× micro, plus 60% CO₂, 130 μMol light after 72 hours growth in bioreactors (3 reps/exp).

Table 1 shows sequence identity values from (A) amino acid sequence alignments and nucleotide sequence alignments for poplar eIF-5A3 and eIF-5A from other plants and (B) amino acid sequence alignments and nucleotide sequence alignments for tomato DHS and DHS from other plants.

### DETAILED DESCRIPTION OF THE INVENTION

The present invention is based in part on the finding that overexpressing poplar growth factor 5A (eIF-5A) in transgenic algal cells results in faster algal cell growth and division which in turn leads to an increase in total oil produced per culture. The total oil harvested from transgenic algal cells exceeds that which can be attributed to just an increase in cell number. Accordingly, the present invention is also based in part on the finding that transgenic algal cells overexpressing eIF-5A either alone or in combination with deoxyhypusine synthase (DHS) contain more oil per cell.

The present invention provides transgenic algal cells that overexpress a protein that contains hypusine. The protein that contains hypusine may be eIF-5A. The transgenic algal cells may overexpress enzymes involved in the synthesis, expression, or post-translation of a protein containing eIF-5A, such as DHS and DHH. The transgenic algal cells may overexpress eIF-5A, DHS, DHH, or a combination thereof. The transgenic algal cells of the present invention encompass both prokaryotic and eukaryotic algal cells. The algal cells for producing the transgenic algal cells of the present invention may be any algal cell. The algal cells may be selected from the divisions consisting of Rhodophyta, Chlorophyta, Cyanophyta, and Phaeophyta. Examples of algae include but are not limited to Chlamydomonas reinhardtii, Chlamydomonas moewusii, Chlamydomonas sp. strain MGA161, Chlamydomonas eugametos, and Chlamydomonas segnis belonging to Chlamydomonas; Chlorella vulgaris belonging to Chlorella; Senedesmus obliguus and Scenedesmus acutus belonging to Senedesmus; Dunaliella tertrolecta belonging to Dunaliella; Anabaena variabilis ATCC 29413 belonging to Anabaena; Cyanothece sp. ATCC 51142 belonging to Cyanothece; Synechococcus sp. PCC 7942 belonging to Synechococcus; and Anacystis nidulans belonging to Anacystis.

The algal cells of the present invention may be transformed with an exogenous nucleic acid encoding eIF-5A, DHS, DHH, or a combination thereof. The eIF-5A, DHS, and DHH may be from any source. The source of eIF-5A, DHS, and DHH may be a plant, fungus, or animal source. The plant may be Arabidopsis thaliana (Atl), alfalfa, banana, Carnation, canola, corn, lettuce, rice, potato, poplar, tomato, or tobacco. There may be different isoforms of a plant eIF-5A. For example, Table 1 shows four different isoforms of tomato eIFA5, 5 different isoforms of potato eIFA5, 4 different isoforms of poplar eIFA5, etc. The fungus may be yeast, mold, slime mold, or Neurospora crassa.

The eIF-5A may be from various sources and comprise an amino acid sequence that has at least 60%, 65%, 70%, 75%, 80%, 85%, 90%, 95%, 96%, 97%, 98%, or 99% sequence identity to SEQ ID NO: 4. The eIFA may be poplar eIFA isoform 3 (eIF-5A3) and may comprise SEQ ID NO: 3 or a functional fragment thereof. eIF-5A may have at least 85% sequence identity with SEQ ID NO: 4, as determined by sequence alignment programs using default parameters.

DHS may be from various sources and comprise an amino acid sequence that has at least 45%, 50%, 55%, 60%, 65%, 70%, 75%, 80%, 85%, 90%, 95%, 96%, 97%, 98%, or 99% sequence identity to SEQ ID NO: 4. DHS may comprise SEQ ID NO: 6 or a functional fragment thereof. DHS may have at least 85% sequence identity with SEQ ID NO: 6, as determined by sequence alignment programs using default parameters.

DHH may be from various sources and comprise an amino acid sequence that has at least 45%, 50%, 55%, 60%, 65%, 70%, 75%, 80%, 85%, 90%, 95%, 96%, 97%, 98%, or 99% sequence identity to SEQ ID NO: 8. DHH may comprise SEQ ID NO: 8 or a functional fragment thereof. DHH may have at least 85% sequence identity with SEQ ID NO: 8, as determined by sequence alignment programs using default parameters.

The nucleic acid encoding eIF-5A, DHS, or DHH may be introduced into algal cells using a construct. The nucleic acid encoding eIF-5A, DHS, or DHH may be in a construct. The construct may comprise the nucleic acid encoding eIF-5A, DHS, or DHH operably linked to a regulatory element. The regulatory element may be a promoter that controls the expression of eIF-5A, DHS, or DHH. The promoter may be a Saccharomyces cerevisiae glycolysis enzyme promoter.

Other regulatory elements that may be included on the construct include terminator, marker for selecting the desired cell, enhancer sequences, response elements or inducible elements that modulate expression of a nucleic acid sequence. The choice of regulatory element to be included in a construct depends upon several factors, including, but not limited to, replication efficiency, selectability, inducibility, desired expression level, and cell or tissue specificity.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

The choice of vector and/or expression control sequences to which nucleic acid encoding eIF-5A, DHS, or DHH is operably linked depends directly on the functional properties desired, e.g., protein expression, and the host cell to be transformed. A vector contemplated by the present invention is at least capable of directing the replication and preferably also expression, of the structural gene included in the recombinant DNA molecule in algal cells.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as an algal cell, transformed therewith. Such replicons are well known in the art. In addition, vectors that include a prokaryotic replicon may also include a gene whose expression confers a detectable marker such as a drug resistance. Vectors that include a prokaryotic replicon can further include a prokaryotic or bacteriophage promoter capable of directing the expression (transcription and translation) of the coding gene sequences in an algal cell.

Transformation of algal cells with a recombinant DNA molecule of the present invention is accomplished by well known methods that typically depend on the type of vector used and host system employed. With regard to transformation of algal cells, electroporation and salt treatment methods may be employed. The constructs may also be introduced into the algae by other standard transformation methods, such as for example, vortexing cells in the presence of exogenous DNA, acid washed beads, polyethylene glycol, and biolistics.

The transgenic algal cells of the present invention may be used to produce oil. The transgenic algal cells may be grown in a bioreactor under conditions for a sufficient time to produce oil. The oil may be harvested from the cells by methods known in the art. The oil from the transgenic algal cells may be processed into biodiesel fuel.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1

### Transgenic Algae

### Algae Culture

Scenedesmus acutus (S.a.) and Chlorella vulgaris (C.v.) cells were grown and maintained on solidified BBM media (Stein (1973) (Ed.) Handbook of Phycological methods. Culture methods and growth measurements. Cambridge University Press) in (100×10)-mm Petri plates in a plant growth incubator with 16-h light (100 mmol m⁻² s⁻¹ photosynthetically active radiation)/8 hour dark cycles at 21° C. Transgenic line screens were grown in a Plant Growth Chamber in 25-mm glass test tubes containing liquid BBM media with 16-h light (100 μmol m⁻² s⁻¹ photosynthetically active radiation)/8-h dark cycles, at a temperature of 21° C. on a shaker at 120 rpm. Cells were diluted to an OD600 of 0.01 and placed back on the shaker to determine if the transgenic lines exhibited accelerated growth rates. Growth rate was measured as the OD 600 after 10 days on the shaker.

CO₂ enrichment experiments were initially performed on cultures that were grown in capped 25-mm glass test tubes in a growth chamber with 100 μmol m⁻² s⁻¹ photosynthetically active radiation for 24 h at a temperature of 21° C. CO₂ (100%) was bubbled to each individual test tube through Tygon tubing fitted into the cut end of a 1 cc syringe connected to a 25 gage needle that was placed with the tip on the bottom of each test tube.

Small-scale bioreactors were developed which consisted of a 200-ml glass square jar (Kimax) with a #3 rubber stopper fitted into each neck. The stoppers had 2 holes, one fitted with a cut off 1-cc syringe into which the Tygon tubing providing CO₂ was inserted, and a second hole fitted with 3-cm of the plugged end of a 1-ml plastic pipette which includes the cotton plug (Fisher Scientific Canada). This was used as a vent to prevent pressure build-up in the reactor. Bioreactors were initiated with 20 ml of algae cells at an OD 600 of 4.0. Jars were placed in a plant growth chamber on a rotary shaker at 70 rpm under 24 hour light at 130 μMol and at 21° C. Carbon enrichment was achieved by mixing air flowing at 3 L/min and 100% CO₂ flowing at 2 L/min, resulting in approximately 60% CO₂ enrichment.

### Plasmid Constructs and Bacterial Strains

1. pBI-PGKF5A construct (PF)

The poplar eIF-5A3 cDNA nucleotide sequence is set forth in SEQ ID NO: 3 and the amino acid sequence is set forth in SEQ ID NO: 4. The translation start codon starts at nucleotide 48 and stop codon starts at nucleotide 525. A Saccharomyces cerevisiae glycolysis enzyme promoter, PGK1, was amplified by PCR with primers: upstream 5′-GTCTACAGGCATTTGCAAGAATTACTCG-3′ (SEQ ID NO: 9) with a SalI restriction site and downsteam 5′-GGATCCTGTTTTATATTTGTTGTAAAAAGTAG-3′ (SEQ ID NO: 10) with BamHI restriction site (Kong (2006) Biotechnol. Left 28: 2033-2038). The PCR product of PGK1 promoter was ligated to a pBI101 vector with SalI and BamHI sites, designated pBI-PGK.

Four distinct full-length PdeIF-5A cDNAs, designated PdeIF-5A1, PdeIF-5A2, PdeIF-5A3 and PdeIF-5A4, were isolated by screening a Populus deltoides leaf cDNA library using AteIF-5A1 cDNA. Leaf mRNA was isolated using a Qiagen kit according to manufacturer's instructions. The cDNA library was prepared using the Stratagene ZAP Express cDNA Synthesis Kit and ZAP Express cDNA Gigapack III Gold Cloning Kit according to manufacturer's instructions. The GenBank accession numbers for PdeIF-5A1, PdeIF-5A2, PdeIF-5A3 and PdeIF-5A4 are FJ032302, FJ032303, FJ032304 and FJ032305, respectively. PdeIF-5A3 full-length cDNA including 5′- and 3′-UTR in pBK-CMV vector was digested with BamHI and Sad restriction enzymes. The GUS gene in pBI-PGK was also removed by BamHI and Sad restriction enzyme digestions. The pre-digested PdeIF-5A3 cDNA was then ligated to the pre-digested pBI-PGK vector to form pBI-PGKF5A(PF). The final construct of PF contains PGK1-promoter:PdF5A3-cDNA:Nos-terminator (SEQ ID NO: 1). PF vector was introduced into Agrobacterium tumefaciens GV3101 by electroporation.

The nucleotide sequence of the pPGK:PdF5A3cDNA-tNos construct is set forth in SEQ ID NO: 1. The PGK1 promoter region is in nucleotides 1 to 737. The middle region is poplar eIF-5A3 full length cDNA (including 5′- and 3′-UTR) sequence (nucleotides 738 to 1832). The remaining region is the Nos terminator (nucleotides 1562 to 1832).

2. pBI-PGKFD Construct (FD)

The tomato DHS nucleotide coding sequence is set forth in SEQ ID NO: 5 and the amino acid sequence is set forth in SEQ ID NO: 6. PGK1-promoter plus TDHS (tomato deoxyhypusine synthase) cDNA coding sequences from Solanum lycopersicum plus TEF1-terminator was subcloned into a pBluescript (pBS-KS) vector. PGK1 promoter was amplified by PCR with primers: upstream 5′-AAGCTTAGGCATTTGCAAGAATTACTCG-3′ (SEQ ID NO: 11) with HindIII restriction site and downsteam 5′-ATCGATTGTTTTATATTTGTTGTAAAAAGTAG-3′ (SEQ ID NO: 12) with XhoI restriction site. TDHS was cloned as described in Wang (2001) J. Biol. Chem. 276:17541-17549 and was amplified by PCR with upstream primer 5′-CTCGAGATGGGAGAAGCTCTGAAGTACAG-3′ (SEQ ID NO: 13) with XhoI restriction site and downsteam primer 5′-GGATCCTCAAACTTGGCACCTTATCTGGG (SEQ ID NO: 14) with BamHI restriction site. TEF1 terminator was amplified by PCR from a yeast pFA6a-kanMX6 (Longtine (1998) Yeast 14: 953-961) vector with upstream primer 5′-GGATCCTCAGTACTGACAATAAAAAGATTCTTG (SEQ ID NO: 15) with BamHI restriction site and downsteam primer 5′-ATCGATATCGATACTGGATGGCGGCGTTAGTATCG-3′ (SEQ ID NO: 16) with ClaI restriction site. PGK1 promoter, TDHS cDNA, and TEF1 terminator were digested with restriction enzymes and subcloned into a pBS-KS vector.

PGK1:TDHS:TEF1 construct was digested with HindIII and ClaI from pBS-KS vector. PGK1:PdF5A was amplified by PCR with upstream primer 5′-ATCGATAAGAATTACTCGTGAGTAAGG-3′ (SEQ ID NO: 17) with ClaI restriction site and downsteam primer 5′-GAGCTCTTTTTTTTTTTTTTTTTT-3′ (SEQ ID NO: 18) with Sad restriction site, and pBI-PGKF5A as a template. The PCR fragment was then digested with ClaI and SacI. pBI101 was digested with HindIII and Sad vector to remove GUS gene. Both PGK1:TDHS:TEF1 (SEQ ID NO: 2) and PGK1:PdF5A3 were then ligated to the pre-digested pBI101 to form pBI-PGKFD. pBI-PGKFD contains PGK1:TDHS:TEF1 and PGK1:PdF5A3:Nos. pBI-PGKFD was introduced into Agrobacterium tumefaciens GV3101 by electroporation.

The nucleotide sequence of the pPGK:TDHS-tTEF1 construct is set forth in SEQ ID NO: 2. The PGK1 promoter region is in nucleotides 1 to 733. The middle region is poplar DHS coding sequence (nucleotides 734 to 1879). The highlighted region is the TEF1 terminator (nucleotides 1880 to 2126).

### Transformation of Algae

S.a. and C.v. were transformed according to Kumar (2004) Plant Science 166:731-738, with the following changes. BBM was used as the growth media. Agrobacterium cells were grown in 2×YT media at 28° C. overnight. G418 was used as a selection agent instead of the antibiotic Kanamycin. Transgenic algae colonies appeared on selection media 7-10 days after transformation. Fifty colonies were selected and streaked two times onto fresh selection plates for confirmation of resistance to G418.

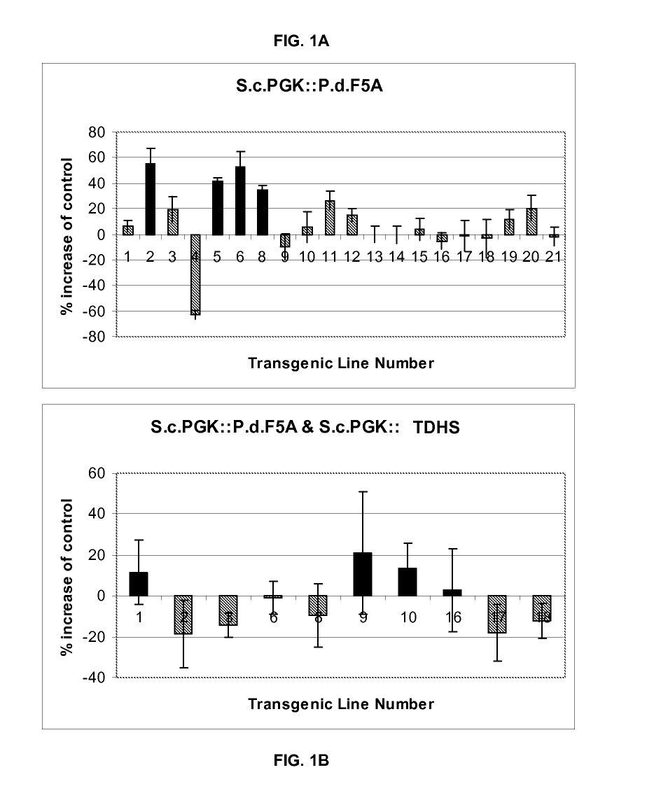

Genetically engineered S.a. and C.v. lines were generated which exhibited overexpression of PdeIF-5A (eIF-5A) alone or in combination with TDHS. Transgenic algae colonies appeared on selection plates 7-10 days after infection with Agrobacterium. As and example, twenty transgenic lines were chosen and analysed after 4 days of growth in liquid culture to identify lines with enhanced growth compared to WT lines without enhanced eIF-5A expression. Of the 20 lines tested, 12 lines with overexpression of eIF-5A under the control of the PGK1 promoter showed an increase in growth over the control line ranging from 4% to 55% (FIG. 1). Lines transformed with a second construct containing both F5A and DHS both driven by the PGK promoter were also tested and produced only 4 lines that performed better than WT lines with increases in growth that ranged from 3% to 20%. Since these experiments were carried out at different times, the differences in the percent increase could be attributed to different conditions of the starting material or growth conditions during the experiment. Thus, the 4 best lines per construct were identified and used for subsequent experiments.

### Example 2

### Oil Content of Transgenic Algal Cells

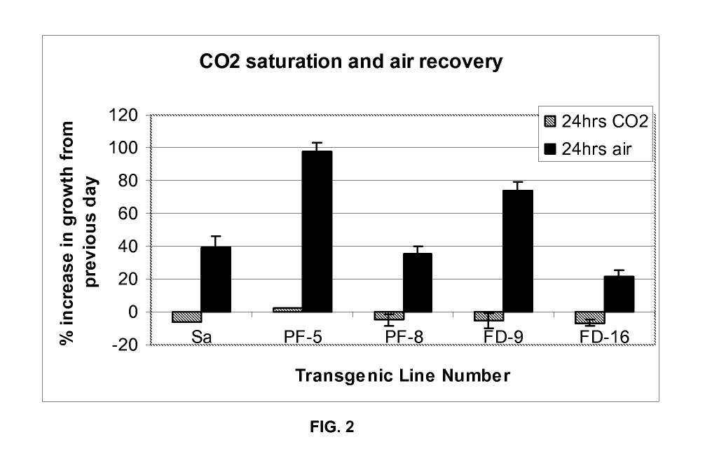

Total lipid content of algal cells was measured using a sulpho-phospho-vanillin reaction (Izaard (2003) J of Microbial Methods 55: 411-418). The goal of producing transgenic algae lines is for their use in a bioreactor to produce oil for biodiesel; thus experiments were designed that mimic the conditions of the bioreactor. Commonly, in bioreactors, 100% CO₂ is bubbled into the algal growth chamber which is subjected to continuous light and constant streaming of algal cells. To simulate these conditions, a CO₂ bubbler was developed for bubbling CO₂ into test tubes containing individual algae lines, thus enabling the testing of multiple lines simultaneously under the same growth conditions. As observed when cultures were initiated with a low cell density, the addition of CO₂ was not necessary and proved to be deleterious to algae growth. Algae cells, cultured for 24 hours with continuous light and 100% CO₂ enrichment did not grow, but remained in a stationary phase. When the CO₂ enrichment was discontinued and air was bubbled into the culture, growth resumed, with much higher growth rates observed in 2 of the 4 transgenic lines tested with PF line 5 exhibiting an increase of 151% over the growth rate of WT (FIG. 2). This experiment demonstrates that algae overexpressing eIF-5A and/or DHS either tolerate a stress episode or recover faster from a stress episode, which in this case was too much CO₂ enrichment resulting in toxic conditions in the growth media.

Small-scale bioreactors were developed. Transgenic lines were screened in the bioreactors under CO₂ enrichment conditions and with increased macronutrient levels [Phosphorous (P), Potassium (K), Calcium (Ca), Magnesium (Mg) and Sulphur (S)]. Conventional algae growth occurs in media such as BBM. Both control and transgenic algae cultures grow faster and produce more oil when grown in media with increased macronutrient levels (4×) and increased micronutrient levels (2×, data not shown).

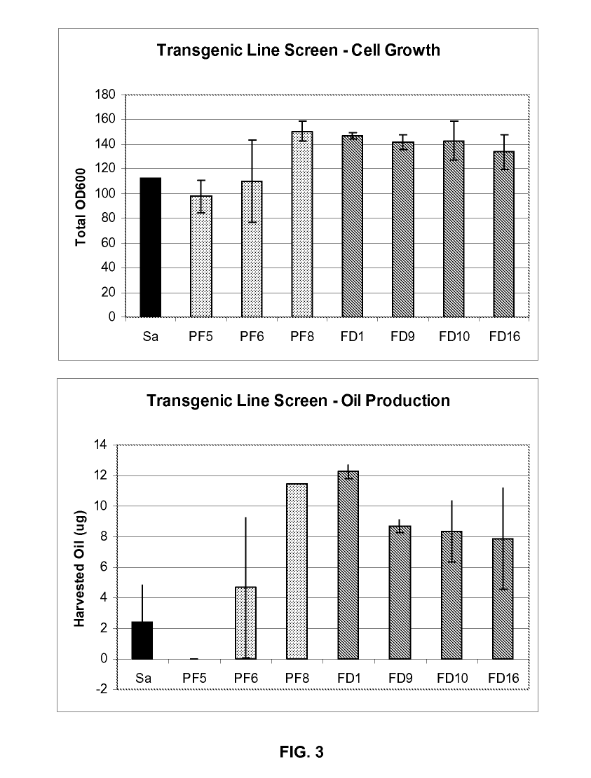

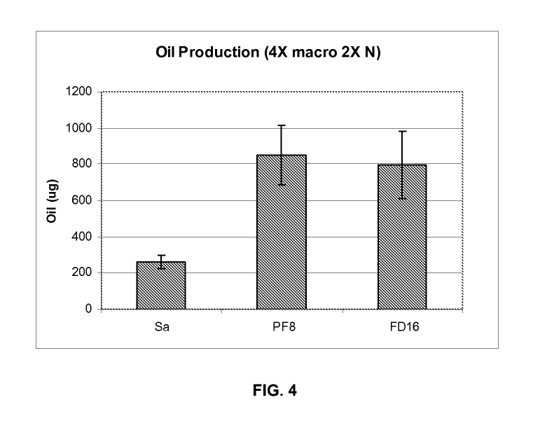

Thus, transgenic lines were screened under these conditions. It was found that 1 PGK:F5A line and all 4 of the PGK:F5A-PGK:TDHS lines exhibited increased growth rates, and that each of these lines had increased oil production (244-407% increase) over that produced from the control line (FIG. 3). Two transgenic lines were chosen to further test oil production. Bioreactors were inoculated using lines PGK:F5A line 8 (PF8) and PGK:F5A-PGK:TDHS line 16 (FD16). When grown in 4× macronutrients with 2× micronutrients and 2× nitrogen for 24 hours, both transgenic lines produced significantly more oil (226 and 206% increase over control, respectively) than control lines grown under the same conditions (FIG. 4).

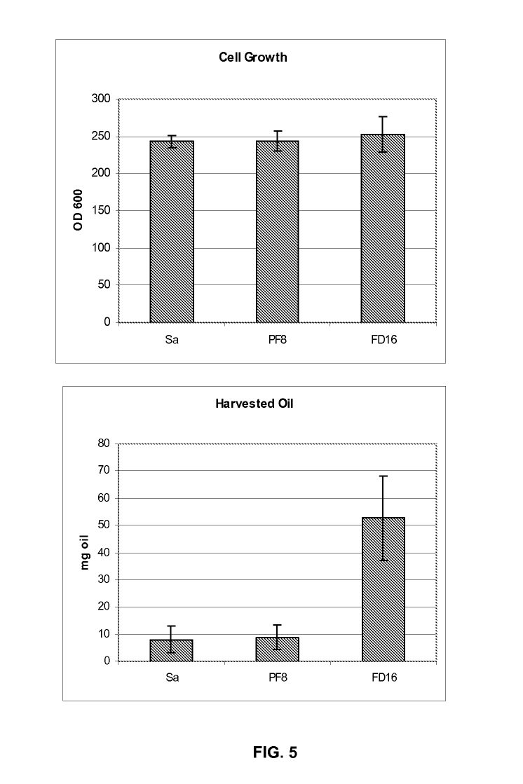

Nutrient levels were further increased to 10× macronutrients, 4× nitrogen and 2× micronutrients, and two lines per construct were grown for a longer period (72 hours) to determine the optimal nutrient levels to produce maximum oil. When grown under these conditions, cell growth was no different between transgenic lines and controls, however, oil production was significantly increased in FD16 (560% increase of control, FIG. 5). These data confirm that overexpression of eIF-5A and/or DHS in algal cells results in increased cell growth and increased oil production.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

1. A transgenic algal cell that overexpresses a protein containing hypusine and produces an increased amount of oil as compared to the amount of oil produced by a corresponding naturally occurring algal cell.

2. The transgenic algal cell of claim 1, wherein the transgenic algal cell overexpresses eukaryotic translation initiation factor 5A (eIF-5A).

3. The transgenic algal cell of claim 2, wherein the eIF-5A comprises an amino acid sequence having at least 85% sequence identity with SEQ ID NO. 3.

4. The transgenic algal cell of claim 2, wherein the cell contains a construct comprising a nucleic acid encoding eIF-5A operably linked to a promoter.

5. The transgenic algal cell of claim 4, wherein the promoter is Saccharomyces cerevisiae glycolysis enzyme promoter.

6. The transgenic algal cell of claim 4, wherein the construct comprises a nucleic acid having a sequence as set forth in SEQ ID NO. 1.

7. The transgenic algal cell of claim 1, wherein the transgenic algal cell overexpresses deoxyhypusine synthase (DHS).

8. The transgenic algal cell of claim 7, wherein the DHS comprises an amino acid sequence having at least 85% sequence identity with SEQ ID NO. 4.

9. The transgenic algal cell of claim 7, wherein the cell contains a construct comprising a nucleic acid encoding tomato DHS operably linked to a promoter

10. The transgenic algal cell of claim 9, wherein the promoter is Saccharomyces cerevisiae glycolysis enzyme promoter.

11. The transgenic algal cell of claim 9, wherein the construct comprises a nucleic acid having a sequence as set forth in SEQ ID NO. 2.

12. The transgenic algal cell of claim 2, wherein the cell further overexpresses DHS.

13. The transgenic algal cell of claim 4, wherein the cell further contains a construct comprising a nucleic acid encoding tomato DHS operably linked to a Saccharomyces cerevisiae glycolysis enzyme promoter.

14. The transgenic algal cell of claim 6, wherein the cell further contains a construct comprising the nucleic acid having the sequence as set forth in SEQ ID NO. 2.

15. A method of producing oil comprising growing transgenic algal cells of claim 1 in a bioreactor under conditions and for a sufficient time to produce oil and harvesting oil from the transgenic algal cells.

16. A method of producing biodiesel fuel comprising growing transgenic algal cells of claim 1 in a bioreactor under conditions and for a sufficient time to produce oil, harvesting oil from the transgenic algae cell, and processing the harvested oil into biodiesel fuel.

17. A method of producing transgenic algal cells that produce an increased amount of oil as compared to the amount of oil produced by corresponding naturally occurring algal cells comprising obtaining a eIF-5A construct comprising the nucleic acid encoding eIF-5A operably linked to a promoter, transforming the algal cells with the construct, cultivating the transformed algal cells under conditions and for a sufficient time to allow growth of the algal cells, and harvesting the algal cells.

18. The method of claim 17, wherein the method further comprising prior to transforming the algal cells, obtaining a DHS construct comprising the nucleic acid encoding DHS operably linked to a promoter and transforming the algal cells with both the DHS and the eIF-5A constructs.


SEQUENCE LISTING (18 sequences)

SEQ ID NO: 1 - DNA, 1832 bp, Artificial sequence
  Sequence of the pPGKPdF5A3cDNA-tNos construct
aggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaggatccaaagaattcgggtattttgtttgttttgtgattgcgactctcttatcaatcgcggccatgtctgacgaggagcagcacttcgagtcaaaagctgatgcgggagcttcgaaaacttaccctcaacaagctggtaccattcgcaagagcggttacattgtcatcaagaatcgcccttgcaaggttgtggaggtttctacctctaaaactggcaagcacggccatgccaaatgtcactttgttgcaattgatatcttcaatggaaaaaagcttgaagatattgttccttcttcccacaactgtgatgttccccatgtcacccgtactgactatcagctgattgatatctcagaggatggatttgtgagcttgctgactgagaatggcaataccaaggatgacctgaggctcccaactgatgagagtctcctctctcagatcaaggatggatttggcgagggtaaagatcttgttgtgactgtgatgtcctccatgggagaggagcagatctgcgccctcaaggacgttggcccgaagtaaattgtaccctgcttcatgcaggcatgcccaaggttgacggctgatagttgtagcttagcaagtggttctatgtgggtttgagaattggtcccgtttcaggaacattccgtactagatgcggccttcccttatatgaagacttgccattatttgagtggcgttttatccgtgatgatatttttgtggttcttggtgtggggaagtttattctgccagtaagactgcatacacttcttggcacctgtggtggtttcaaacaattacagtttttatttccttaaaaaaaaaaaaaaaaaactcgagagtacttctagagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 2 - DNA, 2126 bp, Artificial sequence
  Sequence of the pPGKTDHS-tTEF1 construct
aagcttaggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacactcgagatgggagaagctctgaagtacagtatcatggactcagtaagatcggtagttttcaaagaatccgaaaatctagaaggttcttgcactaaaatcgagggctacgacttcaataaaggcgttaactatgctgagctgatcaagtccatggtttccactggtttccaagcatctaatcttggtgacgccattgcaattgttaatcaaatgctagattggaggctttcacatgagctgcccacggaggattgcagtgaagaagaaagagatgttgcatacagagagtcggtaacctgcaaaatcttcttggggttcacttcaaaccttgtttcttctggtgttagagacactgtccgctaccttgttcagcaccggatggttgatgttgtggttactacagctggtggtattgaagaggatctcataaagtgcctcgcaccaacctacaagggggacttctctttacctggagcttctctacgatcgaaaggattgaaccgtattggtaacttattggttcctaatgacaactactgcaaatttgagaattggatcatcccagtttttgaccaaatgtatgaggagcagattaatgagaaggttctatggacaccatctaaagtcattgctcgtctgggtaaagaaattaatgatgaaacctcatacttgtattgggcttacaagaaccggattcctgtcttctgtcctggcttgacggatggatcacttggtgacatgctatacttccattctttcaaaaagggtgatccagataatccagatcttaatcctggtctagtcatagacattgtaggagatattagggccatgaatggtgaagctgtccatgctggtttgaggaagacaggaatgattatactgggtggagggctgcctaagcaccatgtttgcaatgccaatatgatgcgcaatggtgcagattttgccgtcttcattaacaccgcacaagagtttgatggtagtgactctggtgcccgtcctgatgaagctgtatcatggggaaagatacgtggtggtgccaagactgtgaaggtgcattgtgatgcaaccattgcatttcccatattagtagctgagacatttgcagctaagagtaaggaattctcccagataaggtgccaagtttgaggatcctcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtatcgat

SEQ ID NO: 3 - DNA, 824 bp, Populus deltoides
ggtattttgtttgttttgtgattgcgactctcttatcaatcgcggccatgtctgacgaggagcagcacttcgagtcaaaagctgatgcgggagcttcgaaaacttaccctcaacaagctggtaccattcgcaagagcggttacattgtcatcaagaatcgcccttgcaaggttgtggaggtttctacctctaaaactggcaagcacggccatgccaaatgtcactttgttgcaattgatatcttcaatggaaaaaagcttgaagatattgttccttcttcccacaactgtgatgttccccatgtcacccgtactgactatcagctgattgatatctcagaggatggatttgtgagcttgctgactgagaatggcaataccaaggatgacctgaggctcccaactgatgagagtctcctctctcagatcaaggatggatttggcgagggtaaagatcttgttgtgactgtgatgtcctccatgggagaggagcagatctgcgccctcaaggacgttggcccgaagtaaattgtaccctgcttcatgcaggcatgcccaaggttgacggctgatagttgtagcttagcaagtggttctatgtgggtttgagaattggtcccgtttcaggaacattccgtactagatgcggccttcccttatatgaagacttgccattatttgagtggcgttttatccgtgatgatatttttgtggttcttggtgtggggaagtttattctgccagtaagactgcatacacttcttggcacctgtggtggtttcaaacaattacagtttttatttccttaaaaaaaaaaaaaaaaaa

SEQ ID NO: 4 - protein, 159 aa, Populus deltoides
MSDEEQHFESKADAGASKTYPQQAGTIRKSGYIVIKNRPCKVVEVSTSKTGKHGHAKCHFVAIDIFNGKKLEDIVPSSHNCDVPHVTRTDYQLIDISEDGFVSLLTENGNTKDDLRLPTDESLLSQIKDGFGEGKDLVVTVMSSMGEEQICALKDVGPK

SEQ ID NO: 5 - DNA, 1146 bp, Solanum lycopersicum
atgggagaagctctgaagtacagtatcatggactcagtaagatcggtagttttcaaagaatccgaaaatctagaaggttcttgcactaaaatcgagggctacgacttcaataaaggcgttaactatgctgagctgatcaagtccatggtttccactggtttccaagcatctaatcttggtgacgccattgcaattgttaatcaaatgctagattggaggctttcacatgagctgcccacggaggattgcagtgaagaagaaagagatgttgcatacagagagtcggtaacctgcaaaatcttcttggggttcacttcaaaccttgtttcttctggtgttagagacactgtccgctaccttgttcagcaccggatggttgatgttgtggttactacagctggtggtattgaagaggatctcataaagtgcctcgcaccaacctacaagggggacttctctttacctggagcttctctacgatcgaaaggattgaaccgtattggtaacttattggttcctaatgacaactactgcaaatttgagaattggatcatcccagtttttgaccaaatgtatgaggagcagattaatgagaaggttctatggacaccatctaaagtcattgctcgtctgggtaaagaaattaatgatgaaacctcatacttgtattgggcttacaagaaccggattcctgtcttctgtcctggcttgacggatggatcacttggtgacatgctatacttccattctttcaaaaagggtgatccagataatccagatcttaatcctggtctagtcatagacattgtaggagatattagggccatgaatggtgaagctgtccatgctggtttgaggaagacaggaatgattatactgggtggagggctgcctaagcaccatgtttgcaatgccaatatgatgcgcaatggtgcagattttgccgtcttcattaacaccgcacaagagtttgatggtagtgactctggtgcccgtcctgatgaagctgtatcatggggaaagatacgtggtggtgccaagactgtgaaggtgcattgtgatgcaaccattgcatttcccatattagtagctgagacatttgcagctaagagtaaggaattctcccagataaggtgccaagtttga

SEQ ID NO: 6 - protein, 381 aa, Solanum lycopersicum
MGEALKYSIMDSVRSVVFKESENLEGSCTKIEGYDFNKGVNYAELIKSMVSTGFQASNLGDAIAIVNQMLDWRLSHELPTEDCSEEERDVAYRESVTCKIFLGFTSNLVSSGVRDTVRYLVQHRMVDVVVTTAGGIEEDLIKCLAPTYKGDFSLPGASLRSKGLNRIGNLLVPNDNYCKFENWIIPVFDQMYEEQINEKVLWTPSKVIARLGKEINDETSYLYWAYKNRIPVFCPGLTDGSLGDMLYFHSFKKGDPDNPDLNPGLVIDIVGDIRAMNGEAVHAGLRKTGMIILGGGLPKHHVCNANMMRNGADFAVFINTAQEFDGSDSGARPDEAVSWGKIRGGAKTVKVHCDATIAFPILVAETFAAKSKEFSQIRCQV

SEQ ID NO: 7 - DNA, 945 bp, Arabidopsis thaliana
atggaatctaatggatcagtttcatcgatggttaacttggagaagtttctctgtgagcgattggtggaccagtcacaaccaatctcggagcgatttagagctctcttctctcttcgcaacttgaaaggcccaggacctcgcaacgctctaatccttgcatccagagactcatctaatttgttagcacatgaagctgcatttgcattgggtcagatgcaagatgctgaagccattcctgctcttgagtcggttcttaatgatatgtctttgcatccaatagtacgacatgaggcagcagaagcccttggagctattggtttggcgggtaatgttaacattttaaagaaaagcttgagctcggatccagctcaggaggttcgggaaacatgtgaattagctctcaaaaggattgaagacatgagtaacgttgatgccgagaaccagtcatcaacgacagagaaatcacctttcatgtctgttgacccagcaggcccagctgcgtctttctcttctgttcaccaactcaggcaagttctcctggatgaaacaaaaggcatgtatgagagatatgctgcactgttcgctctaaggaatcatggtggagaggaagctgtttctgccatagttgattctttgagtgctagtagtgcccttctacgtcacgaggttgcttatgtcttgggtcagttgcaaagtaaaactgctttagctactctaagcaaagtacttagagatgtgaatgagcacccaatggttagacatgaggctgcagaagcgcttggttctattgctgatgaacagagcattgctttgctagaagaattctcaaaggaccctgagccaattgttgcacaaagctgtgaagtagcattgagtatgttggaatttgaaaattccgggaaatcgtttgagttttttttcacgcaagacccgcttgttcactaa

SEQ ID NO: 8 - protein, 314 aa, Arabidopsis thaliana
MESNGSVSSMVNLEKFLCERLVDQSQPISERFRALFSLRNLKGPGPRNALILASRDSSNLLAHEAAFALGQMQDAEAIPALESVLNDMSLHPIVRHEAAEALGAIGLAGNVNILKKSLSSDPAQEVRETCELALKRIEDMSNVDAENQSSTTEKSPFMSVDPAGPAASFSSVHQLRQVLLDETKGMYERYAALFALRNHGGEEAVSAIVDSLSASSALLRHEVAYVLGQLQSKTALATLSKVLRDVNEHPMVRHEAAEALGSIADEQSIALLEEFSKDPEPIVAQSCEVALSMLEFENSGKSFEFFFTQDPLVH

SEQ ID NO: 9 - DNA, 28 bp, Artificial sequence
  Primer
gtctacaggcatttgcaagaattactcg

SEQ ID NO: 10 - DNA, 32 bp, Artificial sequence
  Primer
ggatcctgttttatatttgttgtaaaaagtag

SEQ ID NO: 11 - DNA, 28 bp, Artificial sequence
  Primer
aagcttaggcatttgcaagaattactcg

SEQ ID NO: 12 - DNA, 32 bp, Artificial sequence
  Primer
atcgattgttttatatttgttgtaaaaagtag

SEQ ID NO: 13 - DNA, 29 bp, Artificial sequence
  Primer
ctcgagatgggagaagctctgaagtacag

SEQ ID NO: 14 - DNA, 29 bp, Artificial sequence
  Primer
ggatcctcaaacttggcaccttatctggg

SEQ ID NO: 15 - DNA, 33 bp, Artificial sequence
  Primer
ggatcctcagtactgacaataaaaagattcttg

SEQ ID NO: 16 - DNA, 35 bp, Artificial sequence
  Primer
atcgatatcgatactggatggcggcgttagtatcg

SEQ ID NO: 17 - DNA, 27 bp, Artificial sequence
  Primer
atcgataagaattactcgtgagtaagg

SEQ ID NO: 18 - DNA, 24 bp, Artificial sequence
  Primer
gagctctttttttttttttttttt